(19) United States
(12) Patent Application Publication
(10) Pub. No.: US 2002/0000415 A1
(43) Pub. Date: Jan. 3, 2002

(54) Process for treating juices with activated oxygen

(76) Inventors: Lawrence Gene Costello, Orange, CA (US); Elizabeth S. Jenkins, Groveland, FL (US)

Correspondence Address: LAWRENCE GENE COSTELLO, 720 N. ADELE # 4, ORANGE, CA, 92867, US

(21) Appl. No.: 09/803,067

(22) Filed: Mar. 12, 2001

Related U.S. Application Data
(60) Provisional application No. 60/189,408, filed on Mar. 15, 2000.

(51) Int. Cl. A61L 2/00; A23L 3/34

(52) U.S. Cl. 210/764; 422/28

(57) ABSTRACT

A PROCESS FOR ACHEIVING A 5 LOG REDUCTION (99.9%) IN BACTERIA IN JUICES USING ULTRA VIIOLET GENERATED ACTIVATED OXYGEN GAS. THE JUICE IS IN A HOLDING TANK AND THE GAS IS INTRODUCED INTO THE JUICE USING A SPARGING SYSTEM THAT ALLOWS THE GAS TO BE DELIVERED IN 20 TO 60 MICRON SIZE BUBBLES TO ACHEIVE A GREATER CONTACT AREA BETWEEN THE JUICE AND GAS ALLOWING THE ACTIVATED OXYGEN TO REDUCE BACTERIA FAST AND EFFICIENTLY.

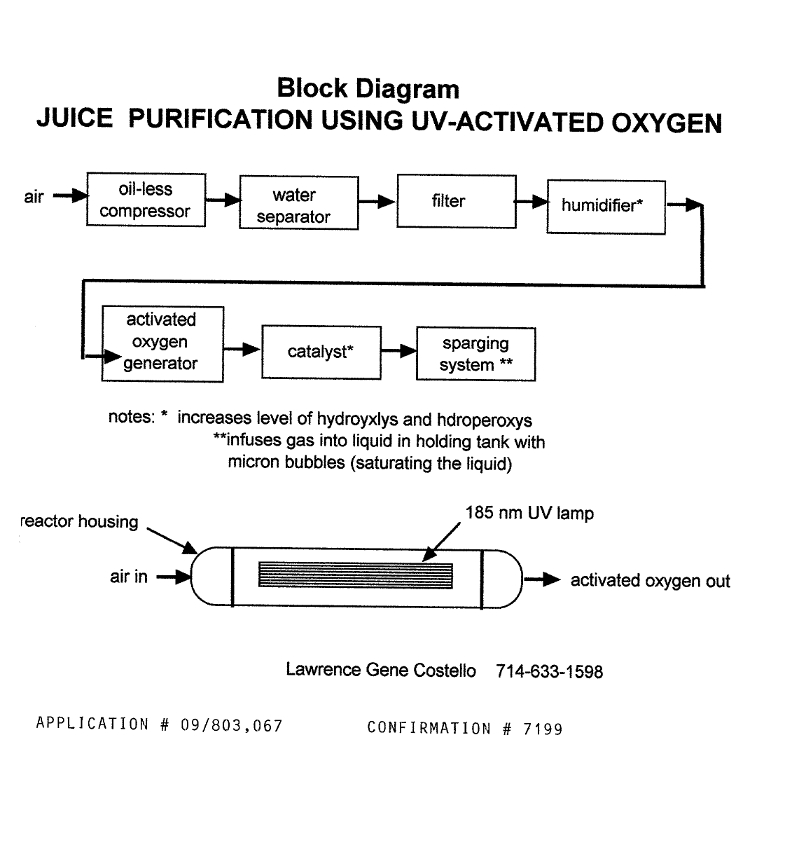

### DESCRIPTION

1. Field of Invention

A. ULTRA VIOLET ACTIVATED OXYGEN HAS BEEN USED SINCE 1982 FOR BACTERIA REDUCTION IN WELL WATER IN THIRD WORLD COUNTRIES TO PROVIDE CLINICALLY PURE WATER

B. ULTRA VIOLET ACTIVATED OXYGEN HAS BEEN USED SINCE 1982 TO REDUCE BACTERIA FROM “GRAY” WATER TO PRODUCE POTABLE WATER

C. ALSO, IN SOME INSTANCES, A HIUMIDIFIER IS USED TO INCREASE THE AMOUNT OF HYDROXYLS AND HYDROPEROXY GENERATED

D. ALSO, IN SOME INSTANCES A SPECIFIC CATALYST IS INTRODUCED INTO THE SYSTEM TO INCREASE THE AMOUNT OF HYDROXYLS AND HYDROPEROXYS.

2. Description of Related Art

A. ULTRA VIOLET ACTIVATED OXYGEN HAS BEEN USED SINCE 1985 TO REDUCE BACTERIA FROM THE EXTERIOR SURFACE AREA OF VEGETABLES (ie. TOMATO, PEPPER, ETC.)

B. ULTRA VIOLET ACTIVATED OXYGEN HAS BEEN USED SINCE 1988 TO REMOVE BACTERIA FROM JUICES (ORANGE, CRANBERRY, APPLE CIDER). THIS WAS DONE ON SMALL SCALE LABORATORY TESTING

C. STARTING IN 1990, TESTING USING ULTRA VIOLET ACTIVATED OXYGEN WAS CONCENTRATED ON APPLE CIDER AND ORANGE JUICE EXCLUSIVELY IN OUR LAB

D. ULTRA VIOLET ACTIVATED OXYGEN IS DEFINED AS:

ASSUMING AMBIENT AIR HAS 21% OXYGEN CONTENT, FOLLOWING IS THE TYPES OF GASSES CREATED BY THE ULTRA VIOLET LAMP (185 NANOMETER) FROM THE 21% OXYGEN:

| | |
|---|---|
| HYDROXY RADICAL | 2% |
| ATOMIC OXYGEN | 0.5% |
| HYDROGEN PEROXIDE | 6% |
| HYDROPEROXY RADICAL | 2% |
| HIGHER PEROXIDES | 7% |
| OZONE | 2% |
| UNKNOWN | 1.5% |

### CROSS REFERENCE TO RELATED APPLICATIONS—NOT APPLICABLE

### STATEMENT REGARDING FEDERALLY SPONSORED RESEARCH—NOT APPLICABLE

### REFERENCE TO MICROFICHE INDEX—NOT APPLICABLE

### SUMMARY

1. BASED ON THE TECHNICAL INFORMATION OBTAINED FROM ACTIVITIES STATED IN “FIELD OF INVENTION” AND“DESCRIPTION OF RELATED ART” AND ON TESTING SINCE 1985 A SPECIFIC PROTOCOL HAS BEEN FORMULATED TO EFFICIENTLY AND AT A LOW COST REDUCE THE BACTERIA FROM JUICE USING ULTRA VIOLET GENERATED ACTIVATED OXYGEN

2. AT PRESENT, PASTEURIZATION (HIGH HEAT) IS THE ONLY F.D.A APPROVED METHOD TO OBTAIN A 5 LOG REDUCTION. UNFORTUNATELY, THE HIGH HEAT DESTROYS ALL THE ENZYMES (AT ANY TEMPERATURE OVER 125 DEGREES F.). ALSO, PASTEURIZATION IS AT LEAST FIVE (5) TIMES MORE EXPENSIVE THAN ULTRA VIOLET ACTIVATED OXYGEN SYSTEMS.

3. THIS ULTRA VIOLET ACTIVATED OXYGEN PROCESS WILL REMEDY THE PROBLEM OF ENZYME LOSS (THERE IS LITTLE OR NO EFFECT ON ENZYMES USING THE ULTRA VIOLET ACTIVATED OXYGEN SYSTEM)

4. IT HAS BEEN OBSERVED THAT SHELF LIFE IS EXTENDED DUE TO REDUCTION OF BACTERIA.

5. THE LOWER INITIAL COST, AND THE LOW MAINTENANCE COST WILL ALLOW EXISTING “MOM AND POP” OPERATIONS TO STAY IN BUSINESS.

What is claimed is:

1. A PROCESS FOR TREATMENT OF JUICES, COMPRISING THE STEPS OF:
A. PUTTING THE JUICE IN A HOLDING, EQUALIZING, BLENDING OR CHILLING TANK
B. SPARGING ULTRA VIOLET GENERATED ACTIVATED OXYGEN INTO THE SOLUTION
C. SPARGING INTO SOLUTION FOR SUFFICIENT PERIOD OF TIME (DEPENDENT ON SIZE OF HOLDING TANK) TO ALLOW THE ACTIVATED OXYGEN GAS TO COME INTO CONTACT WITH THE BACTERIA

2. THE PROCESS ACCORDING TO CLAIM ONE WHEREIN THE PRODUCT IS JUICES (SUCH AS, BUT NOT LIMITED TO APPLE, ORANGE, LIME, GRAPEFRUIT, GRAPE, CRANBERRY, CHERRY, TANGERINE, NECTARINE, PEACH NECTAR, APRICOT NECTAR, PINEAPPLE, RASPBERRY, LEMON, PEAR, ETC. AND ANY MIXTURE THEREOF).
